(19) United States
(12) Patent Application Publication
(10) Pub. No.: US 2014/0256970 A1
(43) Pub. Date: Sep. 11, 2014

(54) REACTOR FOR MULTI-STEP RADIOCHEMISTRY

(71) Applicant: GE HEALTHCARE LIMITED, LITTLE CHALFONT (GB)

(72) Inventor: Alexander Jackson, Amersham (GB)

(73) Assignee: GE HEALTHCARE LIMITED, LITTLE CHALFONT (GB)

(21) Appl. No.: 14/348,351

(22) PCT Filed: Sep. 28, 2012
(86) PCT No.: PCT/US2012/057683

§ 371 Date: Mar. 28, 2014

Related U.S. Application Data
(60) Provisional application No. 61/541,159, filed on Sep. 30, 2011.

(51) Int. Cl. B01J 19/00; C07C 247/08

(52) U.S. Cl. CPC B01J 19/0046; C07C 247/08
USPC 552/1; 422/159

(57) ABSTRACT

A stacked reactor vessel provides two separate reaction vessel compartments for performing chemical reactions and is particularly suited to multi-step chemical reactions and for incorporation into a cassette for automated operation.

### FIELD OF THE INVENTION

The present invention relates to the field of multi-step radiochemistry on automated platforms. More specifically, the present invention is directed to a reactor for click chemistry

### BACKGROUND OF THE INVENTION

The challenge of radiolabelling complex and often expensive biomolecules with fluorine-18 has been highlighted by Kuboyama et al (Bioorganic & Medicinal Chemistry 19 (2011) 249-255). There is a need for radiochemical methodology for labeling biomolecules that are present in the smallest amount possible. One possible solution to this goal is to prepare a radiolabelled synthon (e.g. [¹⁸F]fluoroethylazide and couple this to a biomolecule vector using a fast and high yielding reaction such as the Cu-catalyzed Huisgen ‘click’ reaction. Where the biomolecule is expensive, can only be obtained in small quantities or where high effective specific activity is required, the radiolabelled synthon must be obtained in a chemically and radiochemically pure form prior to coupling with the biomolecule.

Such a process can be performed in a two-step “one pot” process where the biomolecule is coupled to the radiolabelled synthon in a crude reaction mixture which contains synthon precursor compound. It has been shown that yields of the two-step “one pot” process ‘click labelling’ can be low when the process is done in one reactor. This is partly due to the consumption of the biomolecule (e.g. vector-alkyne conjugate where the coupling reaction is Cu-catalyzed Huisgen) by the unlabelled precursor e.g. tosylethylazide. One way around this is to use a two-step process where the labelled e.g. fluoroethylazide is purified (by distillation or chromatography) and is coupled to the alkyne in a second step (Glaser, M. & Robins, E. G. ‘Click labelling’ in PET radiochemistry. Journal of Labelled Compounds & Radiopharmaceuticals 52, 407-414 (2009). Glaser, M. et al. Methods for 18F-labeling of RGD peptides: Comparison of aminooxy [18F]fluorobenzaldehyde condensation with ‘click labeling’ using 2-[18F]fluoroethylazide, and S-alkylation with [18F]fluoropropanethiol. Amino Acids 37, 717-724 (2009). Glaser, M. & Årstad, E. ‘Click labeling’ with 2-[18F]fluoroethylazide for Positron Emission Tomography. Bioconj. Chem. 18, 989-993 (2007).).

It has been proposed that a solid-supported precursor (resin-linker-vector or RLV) for [¹⁸F]fluoroethylazide could be an efficient way to generate this precursor in a clean way without chromatography or distillation. For this approach to work there needs to be some way to heat the reaction of [¹⁸F]fluoride with the RLV. This might require a second reaction heater which could be a simple cartridge heater. It has also been proposed that two step labelling using an RLV could be achieved using the standard FASTlab® reaction heater by using a partitioned reactor (such as that disclosed in Applicant's commonly-assigned patent application entitled “Partitioned Reaction Vessels”, docket no. PH-1170P, filed on even date herewith) and a solid-supported copper catalyst (e.g. Steve Ley et al. Org. Biomol. Chem., 2007, 5, 1562-1568. Steve Ley et al. Angew. Chem. Int. Ed. 2009, 48, 4017-4021). The partitioned reactor approach may require that the fluoride be dried in the presence of the RLV precursor and would also require a modified reaction vessel.

There is therefore a need in the art for a device which can perform a two-step click labeling reaction on an automated synthesizer to allow the radiolabelling of biomolecules in a single reactor with a single heating element whilst requiring small chemical quantities of the biomolecule.

### SUMMARY OF THE INVENTION

The present invention provides a reactor vessel having two reaction vessels. The reaction vessels may be provided in a vertically stacked arrangement such that a heated reactant fluid from the lower reaction vessel can be provided to the upper reaction vessel. What's more, fluid may be reciprocally moved between the first and second reaction vessels for more complete reactions in the second reaction vessel.

In one embodiment, the present invention provides a reactor vessel for an automated synthesizer in which one reaction vessel is seated within a heating well on the synthesizer. In one particular embodiment, the present invention provides a reactor vessel for a FASTlab® synthesizer reaction vessel providing a second reaction compartment mounted directly to the central dip tube luer fitting in the first reaction vessel.

When used in combination with a solid-supported precursor, the present invention will allow two-step radiochemistry to be performed using only one reaction heater. This will also have the advantage that excess precursor from the first step will not be in the same reaction medium during the second step. This could reduce the formation of by-products, increase yields and allow a lower amount of the second precursor used in the second step. This could reduce the cost of goods for the process overall especially if a second precursor is an expensive peptide.

While azide-alkyne ‘click’ radiochemistry is used as an example of a possible application of this process, those of ordinary skill in the art will recognize that the present invention may be applied to other multi-step chemical reactions.

An exemplary embodiment includes a reactor vessel having a first reaction vessel and a second reaction vessel. The first reaction vessel includes a first vessel body defining a first vessel chamber, the first vessel body including a first port a second port, and a third port, each of the first, second, and third ports define a passageway therethrough in fluid communication with the first vessel chamber. The first reaction vessel further includes an elongate dip tube having an elongate tubular body defining a first open end, an opposed second open end, and an elongate dip tube passageway extending in fluid communication therebetween. The dip tube transits the second port in a fluid-tight connection. The second reaction vessel includes a second vessel body defining a second vessel chamber. The second vessel body includes first and second ports, each of the first and second ports define a passageway in fluid communication with the second vessel chamber. The second vessel chamber includes a reactant media therein.

Another exemplary embodiment includes a cassette for performing multi-stage chemical reaction. The cassette includes an elongate manifold including first and second end valves and a plurality of interior valves oriented along a manifold flowpath therebetween. The manifold defines an elongate manifold flowpath between each of the valves. The cassette also includes a reaction vessel of the present invention, at least one pump means supported on a valve, at least one reagent vial holding contents which are directable into the manifold flowpath, and at least one reaction vessel connected across two of the valves.

Yet another exemplary embodiment includes a method for performing a multi-stage chemical reaction having the the steps of:

a) Trapping [¹⁸F]Fluoride on a cartridge;

b) eluting the trapped [¹⁸F]Fluoride with an eluent into a reactor of the present invention via a reaction vessel side arm

c) Adding reaction solvent via a side arm or dip tube and heating the reaction solvent to allow the fluoride complex to dissolve

d) Drawing up the hot fluoride solution through the dip tube to a solid supported precursor, holding the hot fluoride solution at the solid supported precursor, and returning the solution back into the reaction vessel to be heated again

e) Repeating the drawing step until the fluoride incorporation level is acceptable

f) Directing the solution of [¹⁸F] labeled synthon into the reactor or optionally washing off the RLV using clean solvent.

### BRIEF DESCRIPTION OF THE DRAWINGS

FIG. 1 is a schematic of ‘stacked’ dual reactor of the present invention.

FIG. 2 depicts a cross-sectional view of the reactor of FIG. 1, taken through the line 2-2.

FIG. 3 depicts a cassette incorporating the reactor of FIG. 1.

FIG. 4 depicts a generic reaction scheme for chemistry that may be suitable for the ‘stacked’ dual reactor.

FIG. 5 depicts an example of chemistry that is suitable for the ‘stacked’ dual reactor.

FIG. 6 depicts an example of radioiodination chemistry that is suitable for the ‘stacked’ dual reactor.

### DETAILED DESCRIPTION OF THE PREFERRED EMBODIMENTS

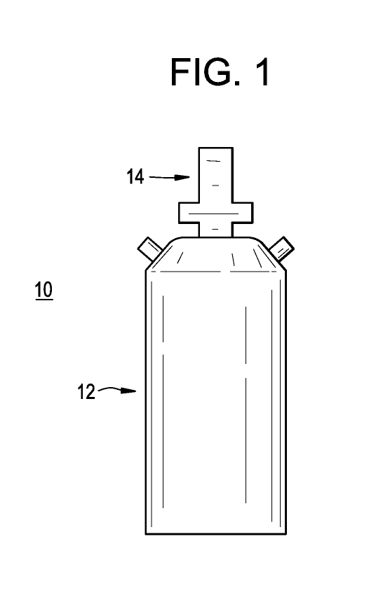

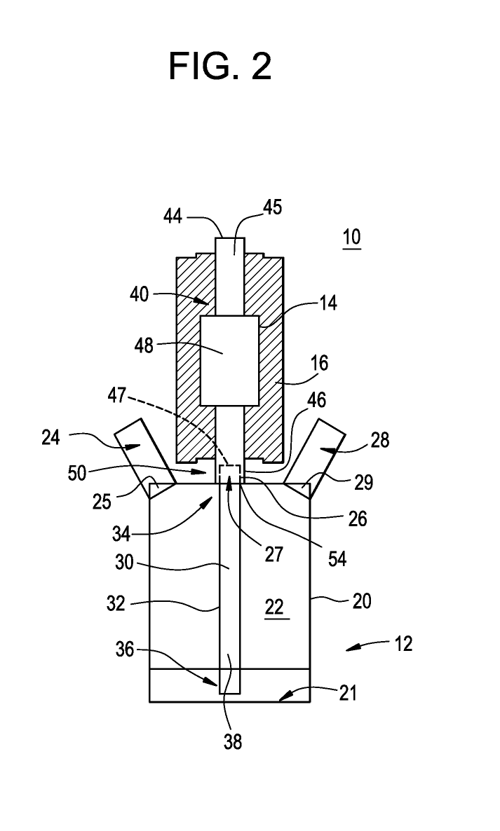

Referring now to FIGS. 1 and 2, the present invention provides a reactor 10 having a first reaction vessel 12 and a second reaction vessel 14. Reactor 10 may be provided on an automated synthesis cassette, such as used for the FASTlab® synthesizer sold by GE Healthcare, Liege Belgium, although the present invention is contemplated to be usefully applied by other synthesis cassettes or for other automated synthesizers having a heater for a reaction vessel of the present invention and able to operate with the reaction vessel in a manner of the present invention. FASTlab® cassettes are disposable cassettes which mate to, and are operated by, the FASTlab® synthesizer. First reaction vessel 12 is desirably sized to fit within a heating well containing a heating element so that reactions within reaction vessel 12 may take place at elevated temperatures and thus provide heated input material to second reaction vessel 14. Second reaction vessel 14 may thus freely extend out from the heating well into standard room conditions. Reactor 10 is desirably made from a suitable polymeric material for withstanding thermal stresses and for withstanding any fluids provided thereinto. Second reaction vessel 14 desirably has an insulation jacket 16 positioned thereabout to help maintain the elevated temperature of the material provided from the first reactor.

First reaction vessel 12 includes a first vessel body 20 defining a first vessel chamber 22 and also includes a first port 24, a second port 26, and a third port 28. Ports defining 24, 26, and 28 define a passageway 25, 27, and 29, respectively, therethrough in fluid communication with first vessel chamber 22. First reaction vessel 12 further supports an elongate dip tube 30 having an elongate tubular body 32 defining a first open end 34, an opposed second open end 36, and an elongate dip tube passageway 38 extending in fluid communication therebetween. Dip tube 30 transits through passageway 27 in a fluid-tight connection with second port 26. Second open end 36 of dip tube 30 is desirably provided in spaced registry with a bottom surface 21 of vessel body 20, although the precise spacing may be dictated by the synthesis process for which reactor 10 supports.

Second reaction vessel 14 includes an elongate second vessel body 40 defining a second vessel chamber 42. Second vessel body 40 includes opposed first and second ports 44 and 46 defining first and second apertures 45 and 47, respectively. Apertures 45 and 47 are in fluid communication with second vessel chamber 42. Second reaction vessel 14 further supports a reactant media 48 in chamber 42. Reactant media 48 may be a frit supporting an RLV for chemically reacting with a fluid provided into chamber 42. First end 34 of dip tube 30 opens in fluid communication with chamber 42 so that fluid provided from first reaction vessel 12 is delivered into chamber 42 through dip tube 30. Similarly, fluid delivered from second reaction vessel 14 to first reaction vessel 12 will be delivered into first chamber 22 from second chamber 42 via dip tube 30.

Reactor 10 is desirably sized so that first reaction vessel 12 fits into the heating well of an automated synthesizer (such as a FASTlab®). In one embodiment of the present invention, second reaction vessel 14 could be a cartridge which incorporates a frit and includes male luer fitting 50 about aperture 47 and a luer cap 52 providing a female luer fitting 54 about aperture 45, similar to an SPE cartridge. This arrangement allows the reaction on the RLV embedded in the frit to be performed without the need for a second reaction heater. This may be possible since a hot solution of fluoride may be passed directly into second reaction vesse114. The RLV cartridge, ie vessel 14, may also be fitted with a thermal insulator 16 to avoid rapid cooling within chamber 42.

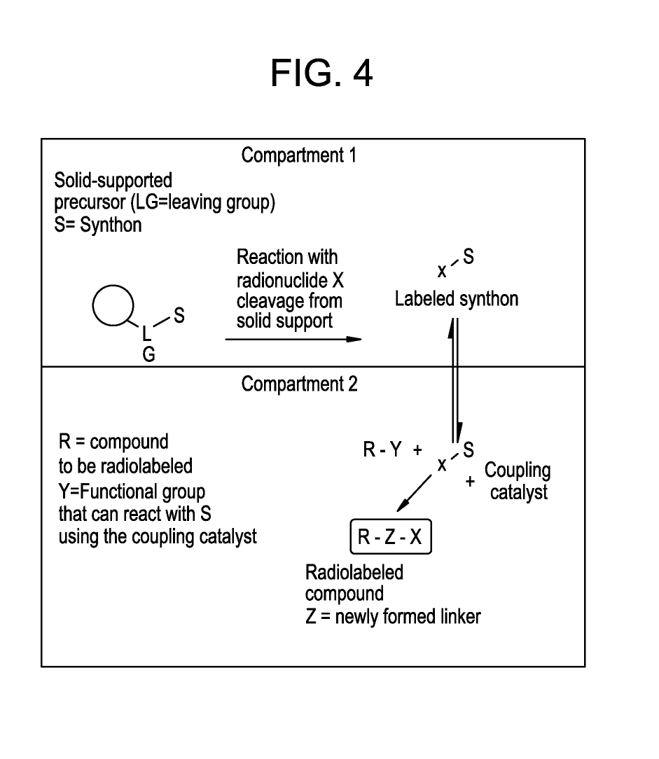

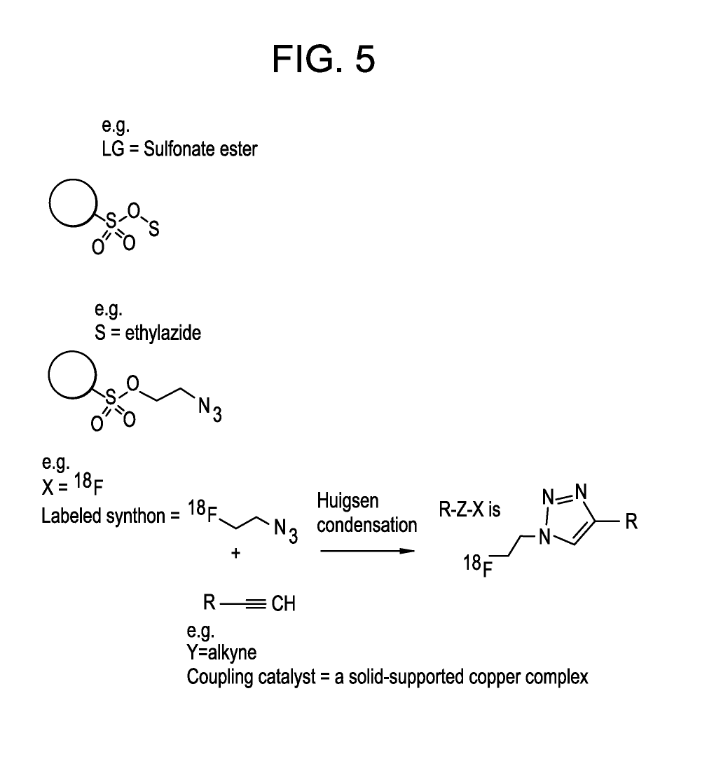

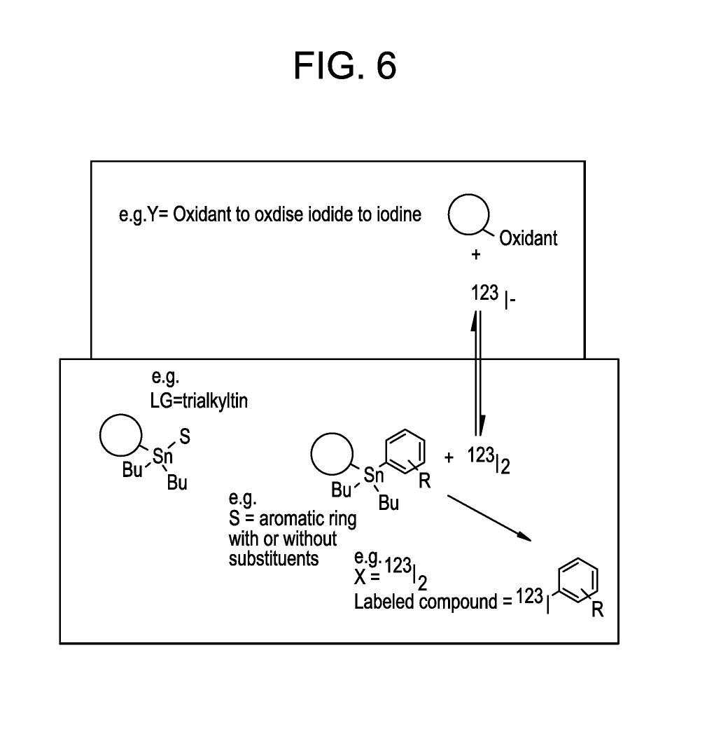

Whilst this idea uses “click chemistry” as an example in FIG. 5, if the concept is successful, the ‘stacked’ reactor could be applied to other synthon based radiochemistry (other synthons and/or other radioisotopes etc. as shown in FIG. 4 and FIG. 6). This arrangement of one reactor stacked on top of another using one heater for both with a thermal insulation jacket may apply to other synthesis processes or automated radiochemistry platforms. It should also be clear to a person skilled in the art that the solid-supported precursor (RLV) 48 may alternatively bear an alkyne functional group instead of an azide functional group. In this instance the second precursor will have an azide functional group instead of the alkyne functional group. The synthon RLV may be designed such that it has low volatility and therefore the volume of the solution in 22 can be reduced by evaporation prior to addition of the second precursor and catalyst as needed.

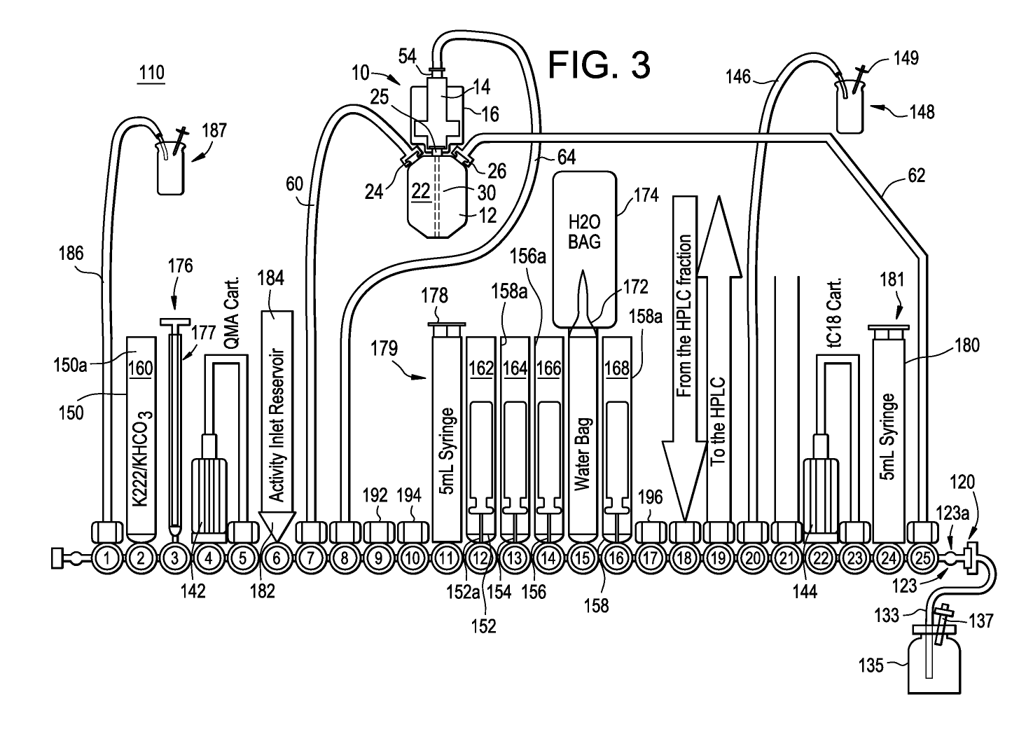

Referring now to FIG. 3, the present invention provides a cassette 110 for performing a multiple-step chemical reaction. Cassette 110 is particularly suitable for performing radiochemistry synthesis methods. Cassette 110 may be formed as a one-use, or disposable, device for synthesizing a compound. Cassette 110 is removably mounted to a synthesis device, such as FASTlab®, so that required connections may be made between cassette 110 and other components, e.g., a source of a radioisotope, dispense vials configured for receiving either product fluid or waste, as well as motive fluid sources.

Cassette 110 desirably includes a polymeric housing (not shown) having a planar major front surface and defining a housing cavity in which an manifold 112 is supported. Cassette 110 includes reactor vessel 10 and vessel ports 24 and 28 are connected in individual fluid communication with valves 7 and 25 via elongate fluid conduits 60 and 62, respectively. Luer fitting 54 is connected to valve 8 via elongate fluid conduit 64. Reactor vessel 10 is sized such that first reaction vessel 12 may be placed within a heating cavity of the synthesizer so that heat may be applied to reaction occurring in chamber 22.

As shown in FIG. 3, cassette 110 is connectable to an HPLC purification system (not shown) such that the synthesizer is able to direct fluid to the HPLC system from valve 19 and then return a purified fluid therefrom back to cassette 110 for additional processing, such as formulation. The return of the purified fluid back to cassette 110 may be provided by connecting an HPLC collected fraction vial to valve 18 via an elongate conduit.

A QMA cartridge 442 is positioned between manifold positions 4 and 5 while a second separations cartridge 444 is positioned between manifold positions 22 and 23. QMA cartridge 442 is used for capture and release of fluoride at the start of the synthesis. While these solid-phase separations cartridges are shown at these locations, the present invention contemplates that solid-phase extraction cartridges may be arranged depending in the requirements of the labeled compound, at positions 17-20 on the manifold to allow purification and processing. Second separations cartridge 444 is used for solvent exchange, or formulation. A length of Tygon™ tubing 146 is connected between manifold valve 21 and a product collection vial 148 in which is dispensed the formulated drug substance. Vial 148 desirably supports a vent needle 149 so as to allow gas within vial 148 to escape therefrom while the vial fills with the product fluid dispensed from cassette 110. While some of the tubings or conduits of the cassette are, or will be, identified as being made from a specific material, the present invention contemplates that the tubings employed in cassette 110 may be formed from any suitable polymer and may be of any length as required.

With continued reference to FIG. 3, manifold 110 includes upstanding hollow vial housings 150, 152, 154, 156, and 158 at valves 2, 12, 13, 14, and 16 respectively. Vial housings 150, 152, 154, 156, and 158 include a cylindrical wall 150a, 152a, 154a, 156a, and 158a defining vial cavities 160, 162, 164, 166, and 168, respectively, for receiving a vial containing a reagent for the reaction. Each reagent vial reagent container includes a container body defining an open container mouth and a container cavity in fluid communication with the container mouth and a pierceable septum sealing said container mouth. Each septum is pierceable by the spike, or cannula, projecting from the manifold valve supporting its respective reagent housing. The present invention contemplates that each container body is adapted to be held in slideable engagement with the cylindrical wall of its respective reagent housing in a first position spaced from the respective spike and a second position in which said respective spike extends through the septum into the container cavity. In the second position the container cavity will be in fluid communication with a valve port of its respective valve so that the reagent may be drawn into the manifold and directed as needed for the radiosynthesis method.

Cassette 110 desirably includes an elongate hollow support housing 170 having a first end supported at valve 15 and an opposed second end supporting an elongate hollow spike 172 extending therefrom. Spike 172 is designed to pierce the septum of a water container 174 which desirably provides a supply of water-for-injection for use in the synthesis process. Cassette 110 further includes a plurality of pumps engageable by the synthesis device in order to provide a motive force for fluids through the manifold. Valves 3, 11, and 24 each support a syringe pump 176, 178, and 180, respectively, in fluid communication with the upwardly-opening valve port and each including a slideable piston reciprocably movable by the synthesizer device. Syringe pump 176 is desirably a 1 ml syringe pump that includes an elongate piston rod 177 which is reciprocally moveable by the synthesis device to draw and pump fluid through manifold 112 and the attached components.

Valve 6 supports an elongate hollow housing 182 having a cylindrical wall 182a defining an open elongate cavity 184. The radioisotope, for example [¹⁸F]fluoride, is provided in solution with H₂[¹⁸O] target water and is introduced at manifold valve 6. Connection of the source of the radioisotope is made to housing 182 prior to the initiation of synthesis. Valve 1 supports a length of tubing 186 extending to a waste collection vial 187 which collects the waste-enriched water after the fluoride has been removed by the QMA cartridge 142. The fluoride will be eluted from cartridge 142, using a solution chosen from but not limited to Kryptofix 2.2.2, potassium carbonate or bicarbonate, tetra-alkyl ammonium salts, potassium mesylate solution, phosphazine base solutions, potassium tert-butoxide from vial housing 150, and delivered to the first reaction vessel 12 via reaction vessel port 24.

Valves 9, 10, and 17 supports luer caps 192, 194, and 196, respectively, thereon in order to seal the upwardly-opening valve port thereof. Syringe pumps 178 and 180 may be a 5 ml syringe pump that includes an elongate piston rod 179 and 181, respectively, which are reciprocally moveable by the synthesis device to draw and pump fluid through manifold 112 and the attached components. Movement of fluid through manifold 112 is additionally coordinated with the positioning of the stopcocks of valves 1-25, the provision of a motive gas at gas ports 121a and 123a as well as by a vacuum, such as that applied to port 120 (possibly through a waste vial 135 connected thereto). The motive gas and the water-for-injection may be pumped through manifold 112 so as to assist in operating cassette 110.

Cassette 110 is mated to an automated synthesizer, such as a FASTlab synthesizer, having rotatable arms which engage each of the stopcocks of valves 1-25 and can position each stopcock in a desired orientation so as to direct fluid flow throughout cassette operation. The synthesizer also includes a pair of spigots, one of each of which insert into ports 121a and 123a of connectors 121 and 123 in fluid-tight connection. The two spigots respectively provide a source of nitrogen and a vacuum to manifold 112 so as to assist in fluid transfer therethrough and to operate cassette 110 in accordance with the present invention. The free ends of the syringe plungers 177, 179, and 181 are engaged by cooperating members from the synthesizer, which can then apply the reciprocating motion thereto within the syringes 175, 178, and 180, respectively. A bottle 174 containing water is fitted to the synthesizer then pressed onto spike 172 to provide access to a fluid for driving compounds under operation of the various-included syringes. Reaction vessel 12 of reactor 10 will be placed within the heating well of the synthesizer and the product collection vial 148 and waste vial 187 are connected. The synthesizer includes a radioisotope delivery conduit which extends from a source of the radioisotope, typically either vial or the output line from a cyclotron, to a delivery plunger. The delivery plunger is moveable by the synthesizer from a first raised position allowing the cassette to be attached to the synthesizer, to a second lowered position where the plunger is inserted into the housing 182 at manifold valve 6. The plunger provides sealed engagement with the housing 182 at manifold valve 6 so that the vacuum applied by the synthesizer to manifold 112 will draw the radioisotope through the radioisotope delivery conduit and into manifold 112 for processing. Additionally, prior to beginning the synthesis process, arms from the synthesizer will press the reagent vials onto their respective cannulas at their manifold valves. Lastly, a conduit 133 is connected to port 120 and spans to a waste vial 135 so that the cavity of vial 135 is in fluid communication with port 120. Waste vial 135 is also pierced by a vent needle 137 which allows gas to pass therethrough but not liquid. A conduit 139 extends from vent 137 to a vacuum port (not shown) on the synthesizer. The synthesis process may then commence.

The present invention further contemplates providing cassette 110 as part of a kit which may be assembled so as to perform a radiosynthesis method. The kit desirably provides cassette 110 with the required lengths of tubing as well as the reagents to be placed in the reagent housings. The kit may further provide the reagent containers positioned within the reagent housings at the first position so that their respective septums are spaced from the underlying spikes of their respective valves.

The labelling procedure utilizing reactor 10 could, by way of illustration and not of limitation, be performed as follows:

1. [¹⁸F]Fluoride from reservoir is directed through valves 6 and 5 into conduit 145 and trapped on QMA cartridge 142. The [¹⁸F]Fluoride is eluted from cartridge 142 with a typical eluent and directed through valves 5, 6, and 7 through conduit 60 and into reaction vessel 12 of reactor 10.

2. The fluoride+eluent is dried (although the drying step may not always be needed depending on the type of eluent used) with nitrogen flow through either dip tube 30 and/or port 28.

3. Reaction solvent is added to chamber 22 via dip tube 30 or port 28 and heated to allow the fluoride complex to dissolve

4. The hot fluoride solution is drawn up through dip tube 30 into the RLV chamber 48, held briefly and then pushed back into reaction chamber 22 to be heated again.

5. Step 4 is repeated until the fluoride incorporation level is acceptable.

6. The solution of [¹⁸F]fluoroethylazide is pushed into reaction vessel 12 or optionally washed off the RLV 48, into 12 using clean solvent.

7. The reactor temperature can be adjusted by the synthesizer as required.

8. Solutions of alkyne and copper catalyst are added to the first chamber 22.

9. The coupling reaction is allowed to complete in the first chamber 22.

10. The reaction mixture is drawn through the RLV frit via dip tube 30 so as to allow excess alkyne to react with RLV-azide.

11. The semi-crude product is withdrawn via dip tube 30 with repeated washing or the reactor/RLV frit, if needed, into a dilution vessel.

12. Purification by SPE cartridge 144 and/or other cartridges that may be present in positions 17-20 of the cassette.

13. Formulation as an injectable solution.

While the particular embodiment of the present invention has been shown and described, it will be obvious to those skilled in the art that changes and modifications may be made without departing from the teachings of the invention. The matter set forth in the foregoing description and accompanying drawings is offered by way of illustration only and not as a limitation. The actual scope of the invention is intended to be defined in the following claims when viewed in their proper perspective based on the prior art.

1: A reactor vessel comprising:
A first reaction vessel comprising a first vessel body defining a first vessel chamber, said first vessel body including a first port a second port, and a third port, each of said first, second, and third ports defining a passageway therethrough in fluid communication with said first vessel chamber, said first reaction vessel further comprising an elongate dip tube having an elongate tubular body defining a first open end, an opposed second open end, and an elongate dip tube passageway extending in fluid communication therebetween, said dip tube transiting said second port in a fluid-tight connection; and
A second reaction vessel comprising a second vessel body defining a second vessel chamber, said second vessel body including first and second ports, each of said first and second ports defining a passageway in fluid communication with said second vessel chamber; said second vessel chamber including a reactant media therein.

2: The reactor vessel of claim 1, wherein said reactant media is a solid-supported precursor.

3: The reactor vessel of claim 1, wherein said reactant media is a solid-supported reagent.

4: The reactor vessel of claim 1, wherein said second open end of said dip tube is positioned adjacent a bottom surface of said first vessel body.

5: The reactor vessel of claim 1, further comprising an insulation material positioned about at least a portion of said second reaction vessel.

6: The reactor vessel of claim 1, wherein said second port of said first reaction vessel matingly engages said first port of said second reaction vessel.

7: The reactor vessel of claim 1, wherein an elongate conduit extends between said second port of said first reaction vessel and said first port of said second reaction vessel.

8: The reactor vessel of claim 2, wherein a frit supports a resin-linker-vector.

9. (canceled)

10: A cassette for performing multi-stage chemical reaction comprising:
An elongate manifold including first and second end valves and a plurality of interior valves oriented along a manifold flowpath therebetween, said manifold defining an elongate manifold flowpath between each of said valves;
the reaction vessel of claim 1;
at least one pump means supported on a valve;
at least one reagent vial holding contents which are directable into said manifold flowpath; and
at least one reaction vessel connected across two of the valves.

11: The cassette of claim 10, wherein
said end valves including at least two valve ports and a stopcock positionable to place either of its respective two valve ports in fluid communication with each other or to fluidically isolate each of its respective valve ports from each other, wherein one of the at least two valve ports opens exteriorly from its respective end valve;
said plurality of interior valves including three valve ports and a stopcock positionable to place at least two said interior valve ports in fluid communication with each other, and wherein two of the valve ports for each valve are in fluid communication with a valve port of an adjacent valve and the third valve port opens exteriorly from its respective interior valve, and
wherein each of said valves supports, in fluid communication with its exteriorly-opening valve port, one of a connector, an elongate open vial housing, a syringe pump, and an elongate open inlet housing, each valve supporting a vial housing further supporting an elongate hollow spike extending into the vial housing;

12: The cassette of claim 11, wherein said first port of said first reaction vessel is connected to a first valve of said manifold via an elongate conduit extending therebetween and said second port of said second reaction vessel is connected to a second valve of said manifold via an elongate conduit extending therebetween.

13-15. (canceled)

16: The cassette of claim 10, wherein said reaction vessel includes a solid-supported catalyst in said second reaction vessel.

17: A method for performing a multi-stage chemical reaction, comprising the steps of:
a) Trapping [¹⁸F]Fluoride on a cartridge;
b) eluting the trapped [¹⁸F]Fluoride with an eluent into the reactor of claim 1 via a reaction vessel side arm
c) Adding reaction solvent via a side arm or dip tube and heating the reaction solvent to allow the fluoride complex to dissolve
d) Drawing up the hot fluoride solution through the dip tube to a solid supported precursor, holding the hot fluoride solution at the solid supported precursor, and returning the solution back into the reaction vessel to be heated again
e) Repeating the drawing step until the fluoride incorporation level is acceptable
f) Directing the solution of [¹⁸F] labeled synthon into the reactor or optionally washing off the RLV using clean solvent.

18: The method of claim 17, further comprising the step of adjusting the reaction temperature in the first reaction vessel.

19: The method of claim 18, further comprising the step of adding solutions of the second precursor and catalyst to the first reactor.

20: The method of claim 17, further comprising the step of completing the coupling reactions in the first reactor.

21: The method of claim 19, further comprising the step of reacting excess second precursor with the solid-supported precursor or RLV in the second reaction vessel.

22: The method of claim 17, further comprising the step of removing the semi-crude product formed by the method to a dilution vessel.

23: The method of claim 17, wherein the fluoride and eluent is dried in the first reaction vessel with nitrogen flow through dip tube and/or left side arm

24: The method of claim 17, further comprising the step of purifying using solid phase cartridges.

25. (canceled)
